(19) United States
(12) Patent Application Publication
(10) Pub. No.: US 2011/0252870 A1
(43) Pub. Date: Oct. 20, 2011

(54) METHOD FOR MONITORING A LINEAR GUIDE

(75) Inventors: Stefan Gluck, Schweinfurt (DE); Frank Benkert, Waigolshausen (DE)

(73) Assignee: SCHAEFFLER TECHNOLOGIES GMBH & CO. KG, Herzogenaurach (DE)

(21) Appl. No.: 13/088,510

(22) Filed: Apr. 18, 2011

(30) Foreign Application Priority Data
Apr. 16, 2010 (DE) 102010015207.2

(51) Int. Cl. G01N 33/30; F16C 33/00; F16C 29/00

(52) U.S. Cl. 73/53.05; 384/8

(57) ABSTRACT

A method for monitoring a linear guide with a carriage that can be displaced along a rail on a lubricated rolling contact is provided having a sensor device for detecting a state of a lubricant for lubricating the rolling contact. In order to allow a quick determination of the state of the lubricant with simple measures, an oscillation time signal is detected by a sensor device from which a characteristic value that increases with increasing degradation of the lubricant is determined by an effective value determination after high-pass filtering. When the characteristic value exceeds a specified threshold value, measures are initiated for improving the lubricating properties.

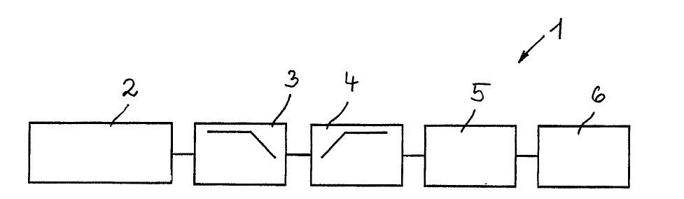

### CROSS-REFERENCE TO RELATED APPLICATIONS

This application claims the benefit of German Patent Application No. 102010015207.2, filed Apr. 16, 2010, which is incorporated herein by reference as if fully set forth.

### FIELD OF THE INVENTION

The invention relates to a method for monitoring a linear guide with a carriage that can be displaced along a rail on a lubricated rolling contact with a sensor device for detecting a state of a lubricant for lubricating the rolling contact.

### BACKGROUND

Linear guides are used for guiding a carriage along a profiled rail. For the construction of the guide contact between profiled rail and carriage, sliding and rolling contacts are used. Rolling contacts are especially preferred due to the conversion of a sliding friction into a rolling friction. Here, roller bodies are arranged either stationary or recirculating between the profiled rail and the carriage. For further reduction of the friction resistance and for the reduction of wear, the roller bodies are supplied with a lubricant, for example, oil or grease that forms a thin lubricating film between roller bodies and running tracks of these bodies. For storing lubricant, for example, a lubricant reservoir could be provided on the carriage, wherein, according to corresponding maintenance specifications, lubricant is dosed from this reservoir onto the rolling contact between roller bodies and running tracks manually or automatically, for example, according to specified operating intervals.

When a lubricating film is formed sufficiently on the rolling contact, the lubricant has a significant influence on the nominal service life of the linear guide.

In order to be able to monitor a sufficient formation of a lubricating film in a linear guide, in DE 10 2006 017 203 A1 it is proposed to use a sensor device for detection, with this sensor device detecting a direct or indirect measure for the presence of a sufficient lubricating film between the roller bodies and a running surface of these bodies. In this way, as a physical measurement parameter, a radiation response is detected by a radiation receiver, with the response following an irradiation on the relevant lubricating area by a radiation source.

### SUMMARY

The object of the invention is the advantageous refinement of a method for monitoring a lubricant on the rolling contact between the roller bodies and their running tracks of a linear guide, in particular, starting from the background described above, using a simple and economical realization of the method using simple components.

The objective is met by a method for monitoring a linear guide with a carriage that can be displaced along a rail on a lubricated rolling contact with a sensor device for detecting a state of a lubricant for lubricating the rolling contact, wherein, through the use of the sensor device, an oscillation time signal is detected, a characteristic value that increases with increasing degradation of the lubricant is determined from the oscillation time signal after a high-pass filtering by an effective-value determination, and if the characteristic value exceeds a specified threshold value, measures are initiated for improving the lubricating properties.

Through the use of the proposed method, all forms of linear guides with lubricated rolling contact, in particular, linear guides constructed as recirculating ball units, can be monitored for a sufficient lubricant film. For the structural construction of linear guides, reference is made to known embodiments as disclosed, for example, in DE 10 2006 017 203 A1.

Measures for improving the lubricating properties could be, for example, an automatic dosing of lubricant and/or an alarm signal. For example, when the threshold value is exceeded, an automated dosing of lubricant could be performed from a storage container that could be arranged, for example, on the carriage or on the profiled rail, wherein a dosing quantity could be set as a function of the measure of the exceeded threshold value. Alternatively or additionally, the alarm signal could be output. In other embodiments, in the case of a very poorly formed or defective lubricating film, the linear guide could be stopped, in that, for example, a drive, such as an electric motor or the like of this guide, for example, of the carriage that can be displaced in a linear fashion on the profiled rail, is included in a control routine of the determination, detection, and evaluation of the signal time response.

The high-pass filtering preferably involves a simple filtering, for example, by a Bessel filter that can be represented in analog or digital and can be preferably of high order, for example, fifth order, so that the construction of the sensor device can be realized easily and by a simple microprocessor. For limiting the frequency range to be detected, low-pass filtering can be performed before the high-pass filtering, so that low-frequency oscillations, for example, sensor resonance and parasitic oscillations can be blanked out. It has proven advantageous when a bandwidth of the oscillation time signal is limited to 16 kHz, advantageously 14 kHz, by the low-pass filtering. Then high-pass filtering is performed for frequencies greater than 8 kHz, advantageously greater than 11 kHz; a relatively narrow frequency band can be detected that lies far below the frequencies of structure-borne acoustic measurements. Here it has been shown surprisingly that for the use of the proposed method in this frequency range, a correlation can be produced between a characteristic value derived from the effective values of an oscillation time signal and the lubricant state. In this way, with simple measures that can be represented in digital and analog, an economical method could be provided on the basis of a corresponding device with a sensor device in which simple sensors can be used that operate with sufficient reliability up to frequencies of 16 kHz, advantageously 14 kHz. Such sensors could be, for example, piezoelectric sensors, sensors produced by microsystem technology, or similar sensors that, for one, can be produced economically and, for another, can be miniaturized. Accordingly, these sensors could be arranged without large structural space requirements advantageously on the carriage of the linear guide.

It has further proven advantageous when the oscillation time signal is detected under defined measurement conditions for comparability of the detected oscillation time signal with oscillation time signals detected under reference conditions. In this way, disruptive influences occurring in this frequency range, for example, artifacts and the like that are typical for the linear guide, can be eliminated in advance. Such disruptive influences can be taken into account, for example, in the threshold value. In the simplest case, this could represent a parameter averaged across the frequency range or could be determined from a set of frequency-dependent parameters. According to the inventive concept, defined measurement conditions are achieved in that the oscillation time signals are detected during at least one measurement travel of the carriage at a known velocity and known load. Here, a constant observance of the velocity across the path of the carriage is preferably provided along the profiled rail; changing velocities, however, could likewise be used for achieving special measurement effects, wherein the velocity profile is specified in a way that can be reproduced and the effective values calculated from this profile supply characteristic values that are compared with threshold values that are fixed taking as a basis the same velocity profiles. A measurement travel of the carriage can here be performed within a short time span, for example, between two working passes of the linear guide integrated into one machine tool, with this time span equaling, for example, less than three seconds and advantageously lying in the range of one second.

The arrangement of the sensor device or the sensor of the sensor device, for example, a piezoelectric sensor, advantageously takes place in the carriage that can be displaced relative to the profiled rail. With respect to its movement plane relative to its transverse movement, the sensor is preferably arranged normal, that is, essentially perpendicular to this with its measurement axis or geometric axis. Alternatively, the sensor could be housed as fixed with respect to these axes perpendicular or parallel to the transverse movement outside or in the movement plane.

### BRIEF DESCRIPTION OF THE DRAWINGS

The invention will be explained in detail with reference to the embodiment shown in the sole FIGURE. This shows a block circuit diagram of a method for determining a characteristic value indicating a degradation of the lubricant of a linear guide.

### DETAILED DESCRIPTION OF THE PREFERRED EMBODIMENTS

The sole FIGURE shows the block circuit diagram 1 for carrying out a method for checking the state of a lubricant on the rolling contact of a linear guide between roller bodies and the associated running tracks of these bodies. In block 2, over a time period of one measurement travel of the carriage, the oscillation time data of the sensor device, for example, the measurement signals of a piezoelectric sensor that is arranged perpendicular to the movement plane of the carriage are read in a data capture device, for example, a volatile or non-volatile memory present in or allocated to a microprocessor. The data advantageously provided from the sensor as analog data and allocated to the oscillations that occur during the measurement travel is here digitized in advance, for example, by an A/D converter. In block 3, the digitized data is low-pass filtered by a filter unit, in that, for example, frequencies above 14 kHz are cut. A corresponding digitally operating filter unit in the form of a low-pass filter could be, for example, a Bessel filter of fifth order, a Butterworth filter or the like. Alternatively, the analog data could be filtered by a discrete low-pass filter constructed from hardware components. Here, the data could be further processed in analog or digitized at this point. In block 4, the data processed up to this point is subjected to a high-pass filter that could be constructed, in turn, if the data is present in analog form, from hardware components or, if the data is digital, from algorithmic computational steps processed in a microprocessor. The high-pass filter can pass oscillation signals remaining from the low-pass filtering and advantageously greater than 11 kHz, so that a frequency range, such as a frequency band between 11 and 14 kHz, is fed to the effective value determination in block 5 and the effective value x_{eff} is determined from the oscillation time signals xᵢ by means of the relationship

$${x}_{eff} = \sqrt{\frac{1}{n} \sum {x}_{i}^{2}}$$

In block 6, the effective value x_{eff} is converted into a characteristic value that is normalized, for example, to a reference magnitude or is processed in some other way and corresponds to a magnitude for the frequency band being used. Observations have shown that the characteristic value increases with increasing degradation of the lubricant, so that in block 5, a comparison with a threshold value could be performed that marks a quality of the lubricant that is still sufficient for forming the lubricating film. If the characteristic value exceeds the threshold value, in the same routine or in another routine, measures are taken for improving or maintaining the lubricating film, for example, activation of a dosing device present in the linear guide for dosing lubricant, output of an alarm signal for operating personnel, and/or initiation of other steps.

1. A method for monitoring a linear guide with a carriage that can be displaced along a rail on a lubricated rolling contact having a sensor device for detecting a state of a lubricant for lubricating the rolling contact, the method comprising: detecting an oscillation time signal using the sensor device, determining a characteristic value that increases with increasing degradation of the lubricant using an effective value determination from the oscillation time signal after a high-pass filtering, and when the characteristic value exceeds a specified threshold value, initiating measures for improving lubricating properties.

2. The method according to claim 1, wherein the measures for improving the lubricating properties are at least one of an automatic dosing of lubricant or an alarm signal.

3. The method according to claim 1, further comprising performing a low-pass filtering before the high-pass filtering.

4. The method according to claim 3, wherein a bandwidth of the oscillation time signal is limited by the low-pass filtering to 16 kHz.

5. The method according to claim 1, wherein the high-pass filtering transmits frequencies greater than 8 kHz.

6. The method according to claim 1, further comprising detecting the oscillation time signals during at least one measurement travel of the carriage at a known velocity and known load.

7. The method according to claim 6, wherein the measurement travel is performed within 3 seconds.

8. The method according to claim 1, wherein the sensor device is fastened on the carriage normal with respect to a movement plane of the carriage.

9. The method according to claim 1, wherein the sensor device is arranged parallel and transverse to a movement direction with respect to a movement plane.

10. The method according to claim 1, wherein the sensor device has a piezoelectric sensor or a sensor fabricated via microsystem technology.
